(19) United States
(12) Patent Application Publication
(10) Pub. No.: US 2022/0194679 A1
(43) Pub. Date: Jun. 23, 2022

(54) MODIFIED ATMOSPHERE PACKAGING FOR SEALED COMPARTMENTS

(71) Applicant: Sage Products, LLC, Cary, IL (US)

(72) Inventor: Lori Beth Smith, Lake in the Hills, IL (US)

(21) Appl. No.: 17/559,602

(22) Filed: Dec. 22, 2021

Related U.S. Application Data
(60) Provisional application No. 63/129,997, filed on Dec. 23, 2020.

(51) Int. Cl. B65D 81/20; B65D 85/07; A61K 8/02; A61Q 17/00

(52) U.S. Cl. CPC B65D 81/2069; A61Q 17/005; A61K 8/0208; B65D 85/07

(57) ABSTRACT

A container includes a compartment defining an inner volume, a plurality of cloth based products positioned within the inner volume of the compartment. The plurality of wipes are at least partially saturated in an aqueous solution. A gaseous mixture fills a remainder of the inner volume. The gaseous mixture includes less than 20% oxygen.

### CROSS-REFERENCE TO RELATED APPLICATIONS

This application claims the benefit of U.S. Provisional Patent Application No. 63/129,997, filed Dec. 23, 2020, which is incorporated herein by reference in its entirety.

### BACKGROUND

The present disclosure relates generally to the field of material packaging. More specifically, the present disclosure relates to contamination prevention within a package.

Intrinsic contamination occurs when a consumable product is contaminated within the product packaging. Intrinsic contamination may occur before the consumable product is used. For example, intrinsic contamination may occur while the consumable product is being stored prior to use.

### SUMMARY

One aspect of the present disclosure relates to a container. The container includes a compartment defining an inner volume and a plurality of cloth based products positioned within the inner volume of the compartment, wherein the plurality of cloth based products are at least partially saturated in an aqueous solution, and a gaseous mixture filling a remainder of the inner volume, and wherein the gaseous mixture includes less than 20% oxygen.

In various embodiments, the compartment includes a liner surrounding the inner volume that substantially prevents ambient air from entering the inner volume until the container is opened. In some embodiments, the aqueous solution includes approximately 2% chlorhexidine gluconate (CHG) solution. In other embodiments, the aqueous solution does not include alcohol. In yet other embodiments, the gaseous mixture includes more than 79% nitrogen. In some embodiments, the gaseous mixture includes less than 15% oxygen and greater than 84% nitrogen. In various embodiments, the cloth based product is a wipe.

Another aspect of the present disclosure relates to a method of manufacturing a product. The method includes providing a container including a compartment defining an inner volume and an opening providing access to the inner volume, providing a plurality of cloth based products within the inner volume of the compartment, wherein the plurality of cloth based products are at least partially saturated in an aqueous solution, providing a gaseous mixture to the inner volume, wherein the gaseous mixture includes less than 20% oxygen, and sealing the opening, thereby containing the gaseous mixture within the container and eliminating access to the inner volume.

In various embodiments, the method also includes providing a liner that surrounds the inner volume and substantially prevents ambient air from entering the inner volume. In some embodiments, the method further includes purging air from within the inner volume using nitrogen prior to providing the gaseous mixture to the inner volume. In other embodiments, the aqueous solution includes approximately 2% CHG solution. In yet other embodiments, the aqueous solution does not include alcohol. In various embodiments, the gaseous mixture includes more than 79% nitrogen. In some embodiments, the gaseous mixture includes less than 15% oxygen gas and greater than 84% nitrogen gas. In other embodiments, the cloth based product is a wipe.

Yet another aspect of the present disclosure relates to a method of cleaning an area of skin. The method includes opening a container including a compartment defining an inner volume, a plurality of cloth based products positioned within the inner volume of the compartment, wherein the plurality of cloth based products are at least partially saturated in an aqueous solution partially including chlorhexidine gluconate (CHG), and a gaseous mixture filling a remainder of the inner volume, wherein the gaseous mixture including less than 20% oxygen, removing a cloth based product, and applying the at least one cloth based product to the area of skin.

In various embodiments, the aqueous solution does not include alcohol. In some embodiments, the cloth based product is a cloth. In other embodiments, the aqueous solution is substantially a 2% CHG solution. In yet other embodiments, the gaseous mixture includes less than 15% oxygen and more than 84% nitrogen.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

The disclosure will become more fully understood from the following detailed description, taken in conjunction with the accompanying figures, wherein like reference numerals refer to like elements, in which:

FIG. 1 shows a schematic view of a consumable product, according to an example embodiment.

FIG. 2 shows a schematic view of a manufacturing system, according to an example embodiment.

FIG. 3 shows a schematic view of a plurality of consumable products, according to an example embodiment.

FIG. 4 shows flow diagram of a manufacturing process, according to an example embodiment.

The foregoing and other features of the present disclosure will become apparent from the following description and appended claims, taken in conjunction with the accompanying drawings. Understanding that these drawings depict only several embodiments in accordance with the disclosure and are therefore, not to be considered limiting of its scope, the disclosure will be described with additional specificity and detail through use of the accompanying drawings.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Intrinsic product contamination may be caused by a variety of organisms, including those that are a part of the Burkholderia Cepacia Complex (BCC). There are roughly twenty different species that make up the BCC, which can proliferate in harsh manufacturing environments and with little nutrition. Further, BCC may survive exposure to otherwise deadly disinfectants. BCC is a respiratory pathogen that does not normally pose a significant risk of serious infection, however, persons with compromised respiratory and/or immune systems (e.g., cystic fibrosis, lung transplant patients, etc.) may be at an increased risk of BCC infection. Thus, in hospitals, care facilities, and anywhere else where persons with compromised respiratory and/or immune systems may be, reducing the risk of intrinsic contamination is desirable.

According to various embodiments, a system and method of reducing the risk of intrinsic contamination in consumable products is disclosed. The disposable products may be cloth based products such as cleaning wipes (e.g., patient preoperative skin preparation wipes). One or more cloth based products may be packaged inside a container that is opened just prior to usage of the cloth based products. The cloth based products may be at least partially saturated in an aqueous solution. The aqueous solution includes 2% Chlorhexidine Gluconate (CHG). The aqueous solution may be alcohol free and/or BZK free. The aqueous solution may have a pH less than 7. The aqueous solution may have a pH of about 4.7 (e.g., between a pH of 4.6 and a pH of 4.8). Further, the aqueous solution may include water.

According to various embodiments, the water in the aqueous solution may be treated to reduce the risk of intrinsic contamination. For example, the water may be subjected to a filtration system and/or UV sterilization. Further, any “dead legs” (i.e., sections of water system piping with low or no flow due to system design and/or operation) may be removed from the manufacturing facility, to reduce the risk of contamination due to stagnant water. Further, some products may be subject to terminal sterilization (i.e., the process of sterilizing a product in its final container) before being used. However, not all consumable products may be amendable to terminal sterilization. For example, terminal sterilization may damage the product and/or reduce the effectiveness of the product. Further, bacterial biofilm may survive terminal sterilization and result in intrinsic contamination. Therefore, additional steps may be taken to further reduce the risk of intrinsic contamination.

Referring generally to the figures, a container is disclosed according to various embodiments. The container includes a compartment defining an inner volume, and a plurality of cloth based products positioned within the inner volume of the compartment, wherein the plurality of cloth based products are at least partially saturated in an aqueous solution, and a gaseous mixture filling a remainder of the inner volume, wherein the gaseous mixture includes less than 20% oxygen.

According to various embodiments, the compartment includes a liner surrounding the inner volume that substantially prevents ambient air from entering the inner volume until the container is opened. The aqueous solution is substantially a 2% CHG solution. According to various embodiments, the aqueous solution does not include alcohol. The gaseous mixture includes greater than 79% nitrogen. The gaseous mixture includes less than 15% oxygen and greater than 84% nitrogen. According to various embodiments, the compartment is collapsible such that when the plurality of cloth based products are removed the compartment may be collapsed such that the inner volume is decreased.

According to various embodiments, a method of manufacturing a product is disclosed. The method includes providing a container including a compartment defining an inner volume and an opening providing access to the inner volume, providing a plurality of cloth based products within the inner volume of the compartment, wherein the plurality of cloth based products are at least partially saturated in an aqueous solution, providing a gaseous mixture to the inner volume, wherein the gaseous mixture includes less than 20% oxygen, and sealing the opening, thereby containing the gaseous mixture within the container and eliminating access to the inner volume.

According to various embodiments, the method further includes providing a liner that surrounds the inner volume and substantially prevents ambient air from entering the inner volume. The method includes purging air from within the inner volume using nitrogen prior to providing the gaseous mixture to the inner volume. According to various embodiments, the aqueous solution is substantially a 2% CHG solution. The aqueous solution may not include alcohol. The gaseous mixture may include greater than 79% nitrogen. According to various embodiments, the gaseous mixture includes less than 15% oxygen gas and greater than 84% nitrogen gas. The compartment may be collapsible such that when the product is removed the compartment may be collapsed such that the inner volume is decreased.

According to various embodiments, a method of cleaning an area of skin is disclosed. The method includes opening a container including a compartment defining an inner volume, a plurality of cloth based products positioned within the inner volume of the compartment, wherein the plurality of cloth based products are at least partially saturated in an aqueous solution partially including chlorhexidine gluconate (CHG), and a gaseous mixture filling a remainder of the inner volume, wherein the gaseous mixture including less than 20% oxygen, removing a cloth based product, and applying the at least one cloth based product to the area of skin.

According to various embodiments, the aqueous solution does not include alcohol. According to various embodiments, the aqueous solution has a pH less than 5. Further, the aqueous solution may be substantially a 2% chlorhexidine gluconate (CHG) solution. According to various embodiments, the gaseous mixture includes less than 15% oxygen and greater than 84% nitrogen.

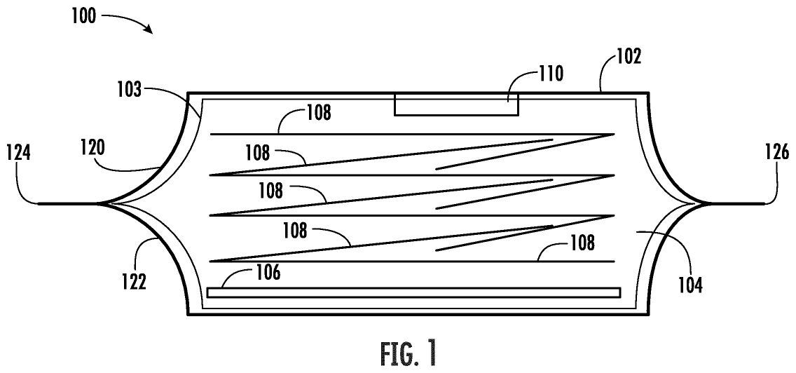

Referring now to FIG. 1, a schematic view of a consumable product 100 is shown, according to an example embodiment. The consumable product includes a container 102 having a compartment that defines an inner volume 104 of the consumable product 100. According to various embodiments, the inner volume 104 is sealed off from the outside environment. For example, the compartment may include a first portion 120 and a second portion 122 that are sealed together at a first end 124 and a second end 126. According to various embodiments, the first portion 120 and the second portion 122 may be integrally formed. For example, before the container 102 is sealed, the container 102 may be generally tubular shaped with a first opening at the first end 124 and a second opening at a second end 126, as will be discussed further with respect to FIG. 3.

According to various embodiments, the container 102 may be made at least partially made of plastic (e.g., high-density polyethylene, linear low-density polyethylene, low density polyethylene, ethylene vinyl alcohol, polylactic acid, thermoplastic elastomer, and aluminum composite foil, etc.). Further, the container 102 may include multiple layers of material. For example, the container 102 may include a first layer that is plastic and a second layer, shown as a liner 103. The liner 103 may substantially surround the inner volume 104. As shown, the liner 103 is positioned inside the container 102, however, it should be appreciated that the liner 103 may surround the outside of the container 102. In some embodiments, the liner 103 surrounds the inner volume 104 such that the liner 103 substantially prevents ambient air from entering the inner volume 104 until the container 102 is opened.

According to various embodiments, the container 102 may be non-rigid. For example, the compartment may be collapsible such that when the plurality of cloth based products are removed the compartment may be collapsed such that the inner volume 104 is decreased. In certain embodiments, the container 102 may be air tight, such that such that a gaseous mixture within the container 102 is sealed off from the outside environment. According to various embodiments, the container 102 is air tight due to a barrier film. For example, the inside and/or the outside of the container 102 may include a barrier film that encompasses the inner volume 104.

The consumable product 100 may further include one or more cloth-based products, shown as, but not limited to, wipes 108 sealed within the container 102. The wipes 108 may be saturated or partially saturated (e.g., only a portion of the wipe is saturated) in an aqueous solution. According to various embodiments, the aqueous solution includes less than 5% Chlorhexidine Gluconate (CHG). For example, the aqueous solution may include 2% Chlorhexidine Gluconate (CHG). According to various embodiments, the aqueous solution may be alcohol free and/or BZK free. According to various embodiments, the aqueous solution may have a pH less than 7. According to various embodiments, the aqueous solution may have a pH of about 4.7 (e.g., between a pH of 4.6 and a pH of 4.8). According to various embodiments, the aqueous solution may include water.

According to the example embodiment shown, the one or more wipes 108 are coupled to one another. For example, as shown, the wipes 108 may be folded such that a portion of a first wipe 108 is folded within a portion of a second wipe 108. In certain embodiments, once the container 102 is opened, removing the first wipe 108 may cause at least a portion of the second wipe 108 to be removed from the container due to the coupling of the first wipe 108 to the second wipe 108. Further, the consumable product 100 includes a support member 106. The support member 106 may provide rigidness to the consumable product 100 to prevent the consumable product 100 from being undesirably folded and/or bent. According to various embodiments, the support member 106 is porous (e.g., a foam) material. In this example embodiment, the support member 106 may also be saturated or partially saturated in the aqueous solution. In this example embodiment, the saturated or partially saturated support member 106 may facilitate maintaining saturation of the wipes 108 during storage of the consumable product 100.

According to various embodiments, the inner volume 104 is filled with a gaseous mixture. The gaseous mixture may be a modified atmospheric gaseous mixture. For example, the gaseous mixture may include nitrogen (N₂), oxygen (O₂), and carbon dioxide (CO₂) gases. Further, in certain embodiments, the gaseous mixture may include helium gas and/or argon gas. In certain embodiments, the gaseous mixture may include more than 79% nitrogen gas. For example, the gaseous mixture may be more than 84% nitrogen gas, more than 89% nitrogen gas, more than 94% nitrogen gas, or more than 99% nitrogen gas. According to various embodiments, the gaseous mixture includes 100% nitrogen gas. According to various embodiments, the gaseous mixture includes less than 20% oxygen gas. For example, the gaseous mixture may include less than 15% oxygen gas, less than 10% oxygen gas, less than 5% oxygen gas, or less than 1% oxygen gas. Further, oxygen gas may not be included in the gaseous mixture.

Microorganisms, such as of the BCC variety, may be aerobic, meaning that they require oxygen to survive. Thus, by providing a gaseous mixture that includes a lesser amount of oxygen gas than the atmosphere (i.e., less than 21% oxygen), the risk of intrinsic contamination may be reduced. For this reason, prior to the container 102 being sealed, a gaseous mixture containing less than 21% oxygen may be provided to the inner volume 104. The container 102 may then be sealed to contain the gaseous mixture within the container 102, thereby reducing the likelihood of intrinsic contamination.

As shown, the consumable product 100 includes an indicator 110. The indicator 110 may be configured to show bacterial and/or oxygen levels within the container 102. For example, the indicator 110 may be in communication with the inner volume 104 of the container 102 such that it may indicate whether the inner volume includes over a threshold percentage of oxygen. For example, the indicator may alter appearance (e.g., change color, display a line, display a text, etc.) in response to an oxygen level being detected over the threshold percentage. The indicator 110 may be a powered oximeter. Alternatively, the indicator 110 may be not include a power source. For example, the indicator 110 may be contain one or more ingredients that react to oxygen levels over the threshold percentage to change the appearance of the indicator 110. In certain embodiments, the indicator 110 may be viewed from outside the container 102 (e.g., through a transparent portion of the container 102). Alternatively, the indicator 110 may be viewed once the container 102 has been opened. In this example, a user of the consumable product 100 may choose to not use the consumable product 100 if the indicator 110 indicates that the container 102 contains over the threshold percentage of oxygen, which may lead to an increased risk of intrinsic contamination.

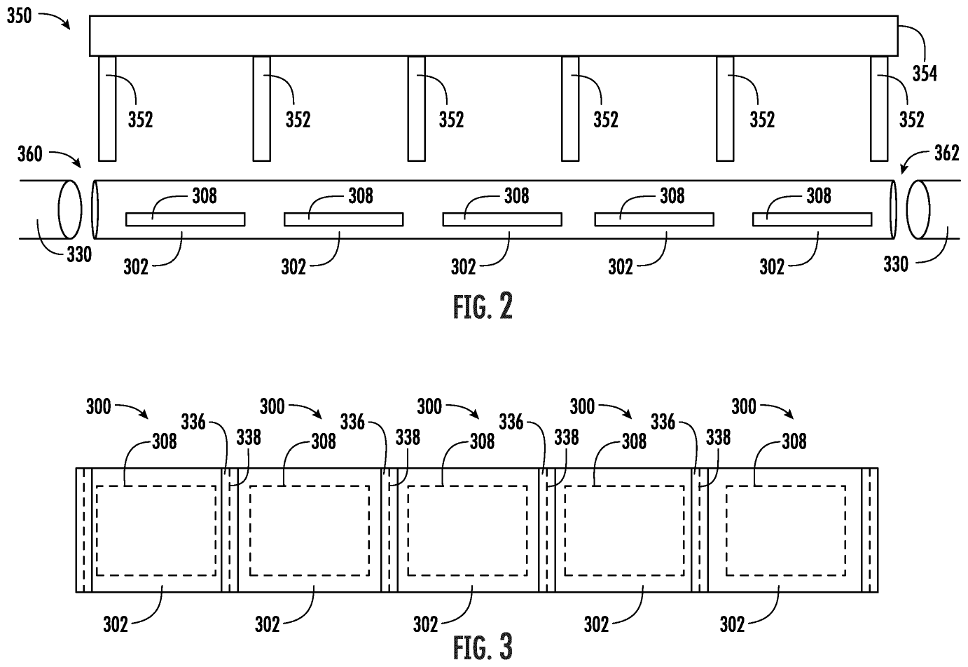

Referring now to FIG. 2, a schematic view of a manufacturing system 350 is shown according to an example embodiment. The manufacturing system 350 may be used during the manufacturing process of the consumable product 100. The manufacturing system 350 is configured to interact with (e.g., fill, seal, cut, etc.) one or more unsealed containers 302. The containers 302 may be the same or similar to the container 102 as described above. For example, the containers 302 include one or more wipe or cloth, shown as wipes 308, which may be the same or similar to the wipes 108. As depicted, the containers 302 are formed of a unitary piece of material, such as plastic. The containers 302 may include a first opening at a first end 360 and/or a second opening at a second end 362. According to various embodiments, the manufacturing system 350 includes at least one gas conduit 330 configured to vacuum air out of the container 302 and/or provide a gaseous mixture to the container 302. For example, a first gas conduit 330 is coupled to the first end 360 of the containers 302 and a second gas conduit 330 is coupled to the second end 362 of the container 302. According to various embodiments, the one or more gas conduits 330 may be configured to provide a gaseous mixture (e.g., any of the gaseous mixtures described herein) having less than 21% oxygen within the containers 302. As shown, the manufacturing system includes a first gas conduit 330 and a second gas conduit 330, however, according to various embodiments, the manufacturing system 350 may only include a single gas conduit 330. For example, the second end 362 may be sealed and the first gas conduit 330 may be used to vacuum the air out of the containers 302 and/or provide the modified atmospheric gas to the containers 302.

After some or all of the containers 302 are filled with a desired gaseous mixture, a sealing device 354 is used to seal the gaseous mixture and the wipes 308 within each individual container 302. For example, a plurality of arms 352 are configured to press against the containers 302 and seal (e.g., via a heat seal) each individual container 302, as is discussed further with respect to FIG. 3. In further embodiments, the arms 352 may be configured to separate (e.g., cut) each individual container 302.

Referring now to FIG. 3, schematic view of a plurality of consumable products 300 is shown according to an example embodiment. The consumable products 300 may be the same or similar to other consumable products described herein, such as the consumable product 100. The consumable products 300 may be manufactured using the manufacturing system 350. The consumable products 300 are coupled to one another via a sealed portion 336. For example the sealed portion 336 may be created when the arm 352 seals the wipes 308 within the container 302. Further, each consumable product 300 may be separated from each other along the separation line 338. For example, the arm 352 may be configured to separate each individual container 302 along the separation line 338 after the wipes 308 are sealed within the container 302.

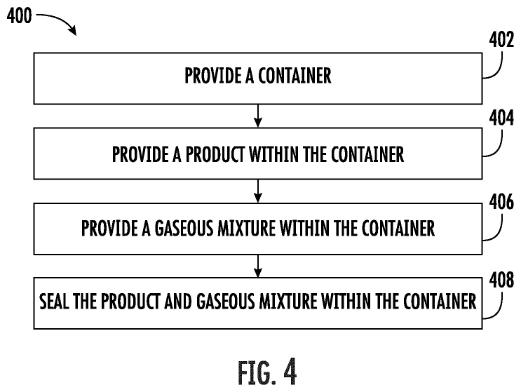

Referring now to FIG. 4, a flow diagram of a manufacturing process 400 is shown, according to an example embodiment. The process 400 may be used to produce consumable products (e.g., the consumable product 100, the consumable product 300, etc.). Further, according to various embodiments, the process 400 may utilize the manufacturing system 350 described above.

At process 402, a container is provided. For example, the container may be a plurality of containers formed from a single piece of material, as described above with reference to FIG. 3, or may be a single container (e.g., the container 102). Alternatively, the container may be formed of two or more pieces of material that are coupled to each other. The container may be generally tubular with at least one opening.

At process 404, a product is provided within the container. For example, a wipe (e.g., the wipe 108, the wipe 308, etc.) may be provided within the opening of the container. The wipe may be saturated in an aqueous solution prior to being provided within the container. In alternative embodiments, process 404 may include providing the aqueous solution within the container to at least partially saturate the wipes. Further, process 404 may include providing a support member (e.g., the support member 106) within the container. According to various embodiments, the product may include other products, such as hygiene products, cosmetics, consumable products, and/or any other product.

At process 406, a gaseous mixture is provided within the container. For example, a gaseous mixture including less than 21% oxygen maybe provided into the opening. The gaseous mixture may include nitrogen gas, carbon dioxide gas, oxygen gas, helium gas, and any other gas described herein. Further, according to various embodiments, process 406 may include vacuuming out some or all of the gas within the container before providing a desired gaseous mixture within the container. For example, a vacuum device may be coupled to an opening in the container such that the vacuum device may draw air out of the container.

At process 408, the product and the gaseous mixture are sealed within the container. For example, a sealing device (e.g., the sealing device 354) may be used to seal the container closed, such that the contents (e.g., the wipe, the gaseous mixture, an indicator, etc.) within the container are sealed off from the outside environment. Process 408 may also include separating one container from another container, as described above with reference to FIG. 3.

Notwithstanding the embodiments described above in reference to FIGS. 1-4, various modifications and inclusions to those embodiments are contemplated and considered within the scope of the present disclosure.

It is also to be understood that the construction and arrangement of the elements of the systems and methods as shown in the representative embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed.

Accordingly, all such modifications are intended to be included within the scope of the present disclosure. Any means-plus-function clause is intended to cover the structures described herein as performing the recited function and not only structural equivalents but also equivalent structures. Other substitutions, modifications, changes, and omissions may be made in the design, operating conditions, and arrangement of the preferred and other illustrative embodiments without departing from scope of the present disclosure or from the scope of the appended claims.

Furthermore, functions and procedures described above may be performed by specialized equipment designed to perform the particular functions and procedures. The functions may also be performed by general-use equipment that executes commands related to the functions and procedures, or each function and procedure may be performed by a different piece of equipment with one piece of equipment serving as control or with a separate control device.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Similarly, unless otherwise specified, the phrase “based on” should not be construed in a limiting manner and thus should be understood as “based at least in part on.” Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent

Moreover, although the figures show a specific order of method operations, the order of the operations may differ from what is depicted. Also, two or more operations may be performed concurrently or with partial concurrence. Such variation will depend on software and hardware systems chosen and on designer choice. All such variations are within the scope of the disclosure. Likewise, software implementations could be accomplished with standard programming techniques with rule based logic and other logic to accomplish the various connection operations, processing operations, comparison operations, and decision operations.

What is claimed is:

1. A container, comprising:
a compartment defining an inner volume;
a plurality of cloth based products positioned within the inner volume of the compartment, wherein the plurality of cloth based products are at least partially saturated in an aqueous solution; and
a gaseous mixture filling a remainder of the inner volume, wherein the gaseous mixture comprises less than 20% oxygen.

2. The container of claim 1, wherein the compartment includes a liner surrounding the inner volume that substantially prevents ambient air from entering the inner volume until the container is opened.

3. The container of claim 1, wherein the aqueous solution includes approximately 2% chlorhexidine gluconate (CHG) solution.

4. The container of claim 3, wherein the aqueous solution does not include alcohol.

5. The container of claim 1, wherein the gaseous mixture comprises greater than 79% nitrogen.

6. The container of claim 1, wherein the gaseous mixture comprises less than 15% oxygen and greater than 84% nitrogen.

7. The container of claim 1, wherein the cloth based product is a wipe.

8. A method of manufacturing a product, comprising:
providing a container including a compartment defining an inner volume and an opening providing access to the inner volume;
providing a cloth based product within the inner volume of the compartment, wherein the cloth based product is at least partially saturated in an aqueous solution;
providing a gaseous mixture to the inner volume, wherein the gaseous mixture comprises less than 20% oxygen; and
sealing the opening, thereby containing the gaseous mixture within the container and eliminating access to the inner volume.

9. The method of claim 8, further comprising providing a liner that surrounds the inner volume and substantially prevents ambient air from entering the inner volume.

10. The method of claim 8, further comprising purging air from within the inner volume using nitrogen prior to providing the gaseous mixture to the inner volume.

11. The method of claim 8, wherein the aqueous solution includes approximately 2% CHG solution.

12. The method of claim 8, wherein the aqueous solution does not include alcohol.

13. The method of claim 8, wherein the gaseous mixture comprises greater than 79% nitrogen.

14. The method of claim 8, wherein the gaseous mixture comprises less than 15% oxygen gas and greater than 84% nitrogen gas.

15. The method of claim 8, wherein the cloth based product is a wipe.

16. A method of cleaning an area of skin, comprising:
opening a container comprising:
a compartment defining an inner volume;
a plurality of cloth based products positioned within the inner volume of the compartment, wherein the plurality of cloth based products are at least partially saturated in an aqueous solution partially comprising chlorhexidine gluconate (CHG); and
a gaseous mixture filling a remainder of the inner volume, wherein the gaseous mixture comprises less than 20% oxygen;
removing a cloth based product; and
applying the at least one cloth based product to the area of skin.

17. The method of claim 16, wherein the aqueous solution does not include alcohol.

18. The method of claim 17, wherein the cloth based product is a cloth.

19. The method of claim 18, wherein the aqueous solution is substantially a 2% CHG solution.

20. The method of claim 16, wherein the gaseous mixture comprises less than 15% oxygen and greater than 84% nitrogen.
